(19) United States
(12) Patent Application Publication
(10) Pub. No.: US 2011/0034530 A1
(43) Pub. Date: Feb. 10, 2011

(54) PROCESS FOR THE PREPARATION OF FIPRONIL AND ANALOGUES THEREOF

(76) Inventors: Teng-Kuei Yang, Taiwan (CN); Erich Widmer, Muenchenstein (CH)

Correspondence Address: BLANK ROME LLP, WATERGATE, 600 NEW HAMPSHIRE AVENUE, N.W., WASHINGTON, DC, 20037, US

(21) Appl. No.: 12/809,705

(22) PCT Filed: Dec. 19, 2008
(86) PCT No.: PCT/IB08/03576

§ 371 Date: Sep. 16, 2010

Related U.S. Application Data
(60) Provisional application No. 61/014,769, filed on Dec. 19, 2007.

(30) Foreign Application Priority Data
Jan. 8, 2008 (FR) 08/50084

(51) Int. Cl. A61K 31/415; C07D 231/44; A61P 33/00

(52) U.S. Cl. 514/407; 548/369.1

(57) ABSTRACT

The present invention relates to a new and efficient process for preparing 5-amino-1-(2,6-dichloro-4-(trifluo-romethyl)phenyl)-4-(trifluoromethylthio)-IH-pyrazole-3-carbonitrile (hereinafter referred to as compound of formula I), which is useful as an intermediate for the antiparasitic agent fipronil, and a process for preparing 5-amino-3-cyano-1-(2,6-dichloro-4-tri-fluoromethylphenyl)-4-trifluoromethyl sulfinylpyrazole (hereinafter referred to as compound of formula II or fipronil). In one aspect, there is provided a process for preparing fipronil comprising: a) a step of reacting CF₃S(═O)ONa with the compound of formula (III) in the presence of a reducing/halogenating agent; and b) a step of oxidizing the compound of formula (I) obtained in step a) in the presence of a selective oxidizing agent, under suitable conditions, wherein the selective oxidizing agent selectively effects oxidation of (I) to the corresponding sulfoxide, Fipronil. In certain exemplary embodiments, the selective oxidizing agent is MHSO₅, wherein M is an alkaline metal cation.

### PRIORITY

The present application claims priority to U.S. Provisional Patent Application No. 61/014,769 filed Dec. 19, 2007 and French Patent Application N° FR 08/50084 filed Jan. 8, 2008; The entire contents of each of these applications are incorporated herein by reference.

### TECHNICAL FIELD

The present invention relates to a new and efficient process for preparing 5-amino-1-(2,6-dichloro-4-(trifluoromethyl)phenyl)-4-(trifluoromethyl-thio)-1H-pyrazole-3-carbonitrile (hereinafter referred to as compound of formula I), which is useful as an intermediate for the antiparasitic agent fipronil, and a process for preparing 5-amino-3-cyano-1-(2,6-dichloro-4-trifluoromethylphenyl)-4-trifluoromethyl sulfinylpyrazole (hereinafter referred to as compound of formula II or fipronil).

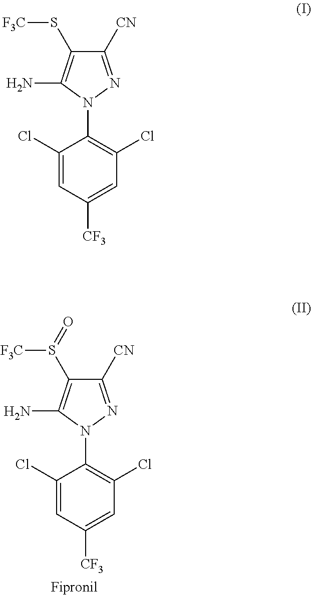

Specifically, the compound of the structural formula (II) can be prepared by reacting CF₃SO₂Na with 5-amino-1-(2,6-dichloro-4-(trifluoromethyl)phenyl)-1H-pyrazole-3-carbonitrile (hereinafter referred to as a compound of formula (III)) in the presence of a reducing/halogenating agent, such as PCl₃ or PBr₃ to prepare the compound of formula (I) with high purity, and then reacting the compound of formula (I) with an oxidizing agent effecting selective oxidation of sulfides to sulfoxides. In certain embodiments, the oxidizing agent is MHSO₅, wherein M is an alkaline metal cation.

In the following, references in brackets ([ ]) refer to the list of references presented after the Examples.

### BACKGROUND OF THE INVENTION

Fipronil is a well-known pesticide that has been extensively used in the agricultural and horticultural industry. Many methods for its preparation have been reported. The most prominent ones consist in chemically transforming the pyrazole precursor of formula III to achieve the introduction of a trifluoromethylsulfinyl group on the unsubstituted position of the pyrazole ring.

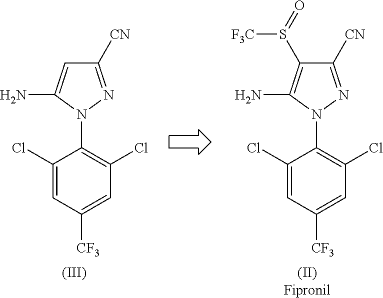

The sulfinylation of heterocyclic compounds, that is the introduction of an RS(═O) group, is typically carried out in one of two conventional ways.

The first one consists in the reaction between a reagent RSX with the heterocyclic compound to give a sulfide-substituted heterocycle which is subsequently oxidized. The difficulties encountered in reported methods include (i) oxidation process difficult to carry out (for example, TFA/H₂O₂ has been used, which renders the process corrosive due to the in situ formation of hydrogen fluoride), and (ii) toxicity of some of the starting reagents (for example, CF₃SCl).

The second one involves direct sulfinylation of the heterocycle. For example, Chinese patent N° CN 1176078C [ref 1] describes a sulfinylation process using a mixture of CF₃SO₂K and CF₃SO₂Na in the presence of a chlorination agent such as POCl₃, PCl₃ or SOCl₂. However, the yields were moderate (74-80%) at labscale. Similarly, EP 0 668 269 [ref 2] describes a one step sulfinylation process involving the reaction of a reagent RS(═O)X with the heterocycle to afford the desired sulfinylated compound. However, the reaction does not always proceed as desired, particularly when the reagent CF₃SO₂H or CF₃SO₂Na is used to carry out the sulfinylation process, since SOCl₂ or phosgene, potentially hazardous, must be used in addition in this case.

A third approach consists in reacting a reagent RX with the S—S bond of a disulfide intermediate, to yield the corresponding sulfide, which is subsequently oxidized. For example, European Patent Publication No. 0374061 [ref 3] and J-L. Clavel et al. in J. Chem. Soc. Perkin I, (1992), 3371-3375 [ref 4] describe the preparation of 5-amino-1-(2,6-dichloro-4-trifluoromethylphenyl)-3-cyanopyrazol-4-yl disulfide, and the further conversion of this disulfide to the pesticidally active 5-amino-1-(2,6-dichloro-4-trifluoromethylphenyl)-3-cyano-4-trifluoromethyl thiopyrazole by reaction with trifluoromethyl bromide in the presence of sodium formate and sulfur dioxide in N,N-dimethylformamide in an autoclave at low pressure (typically 13 bars) at 60° C. However on larger scales the reaction is very exothermic which results in a substantial pressure increase in the vessel and associated operator hazard. Moreover it is necessary to add the trifluoromethyl bromide quickly (generally within 0.5 hour), because the mixture of disulfide, sodium formate, sulfur dioxide and N,N-dimethylformamide has been found to be unstable (typically leading to 55% degradation into unwanted by-products within 2 hours at 50° C.). This requirement for rapid addition of trifluoromethyl bromide is not compatible with the exothermic nature of the reaction.

Thus, the methods known in the art have severe limitations. Specifically, they are often limited in at least one of the following ways:
they use reagents that are too toxic;
they use reagents that are difficult to handle and/or hazardous;
they use somewhat corrosive reagents;
they are difficult to scale up, and thus are not prone to industrial application;
they aim at preparing compounds having a pesticidal activity for use in the agricultural or horticultural industry. Thus, the quality of the product, and particularly its purity, is not necessarily adapted for therapeutic use;
the yields are moderate at labscale.

Thus, there remains a need for developing an efficient and industrially feasible process without these disadvantages.

### SUMMARY OF THE INVENTION

In one aspect, the present invention provides a practical and efficient process for preparing fipronil comprising:

a) a step of reacting CF₃S(═O)ONa with the compound of formula III

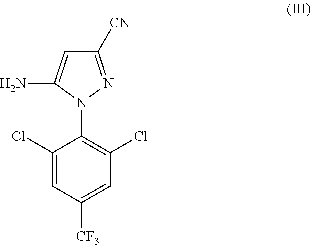

in the presence of a reducing/halogenating agent; and

b) a step of oxidizing the compound of formula I obtained in step a)

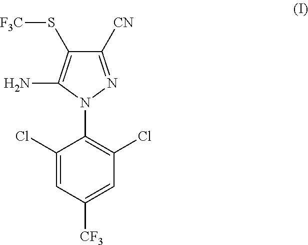

in the presence of a selective oxidizing agent under suitable conditions, wherein the selective oxidizing agent selectively effects oxidation of (I) to the corresponding sulfoxide, Fipronil. In certain embodiments, the selective oxidizing agent is MHSO₅, wherein M is an alkaline metal cation.

In another aspect, the invention provides a practical process for manufacturing an antiparasitic medicament comprising caning out the process according to any one of claims 1-12, and mixing the fipronil obtained by said process with a pharmaceutically acceptable carrier, adjuvant or vehicle.

### DESCRIPTION OF CERTAIN PREFERRED EMBODIMENTS OF THE INVENTION

The present invention aims at overcoming the aforementioned drawbacks. Namely, the present invention seeks to provide an improved, safer or more practical methods for the preparation of antiparasitic agents.

In a first aspect, the invention provides a convenient process for preparing compound of formula I, which is an important intermediate for the synthesis of fipronil.

In a second aspect, the invention provides a safe, high yielding and industrially applicable process for preparing fipronil. The inventive process allows the preparation of fipronil in high purity, which makes it suitable for therapeutic applications.

Thus, in one aspect, there is provided a process for the preparation of the compound of formula I

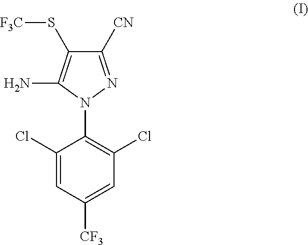

comprising a step of reacting CF₃S(═O)ONa with the compound of formula III

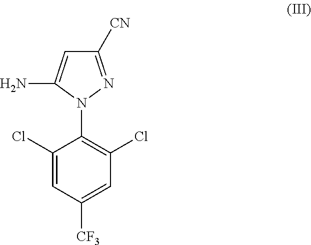

in the presence of a reducing/halogenating agent.

In another aspect, there is provided a process for the preparation of the compound of formula II

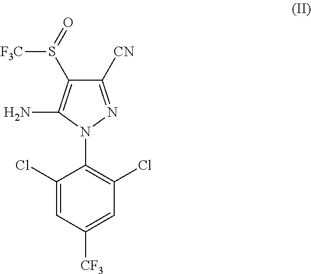

comprising a step of oxidizing the compound of formula I

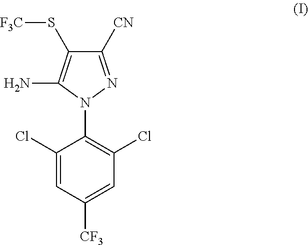

in the presence of a selective oxidizing agent under suitable conditions. In certain embodiments, the selective oxidizing agent is MHSO₅, wherein M is an alkaline metal cation.

In a third aspect, there is provided a process for preparing fipronil comprising:

a) a step of reacting CF₃S(═O)ONa with the compound of formula III

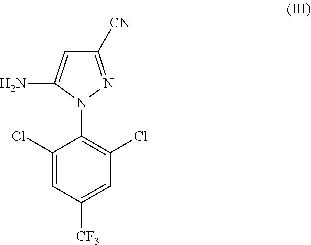

in the presence of a reducing/halogenating agent; and

b) a step of oxidizing the compound of formula I obtained in step a)

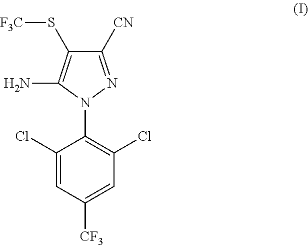

in the presence of a selective oxidizing agent under suitable conditions. In certain embodiments, the selective oxidizing agent is MHSO₅, wherein M is an alkaline metal cation.

In certain embodiments, step b) of the process of the invention is carried such that little or no formation of sulfone (IV) occurs.

In certain embodiments, M represents Li⁺, Na⁺ or K⁺. In certain exemplary embodiments, M is K⁺.

As used herein, the term “reducing/halogenating agent” refers to a halogenating agent that effects sulfenylation of the pyrazole ring of compound III by concomitant reduction at the sulfur atom of CF₃S(═O)ONa.

One important aspect of the invention lies in the discovery that selected halogenating agents, such as PCl₃ or PBr₃ also have the ability to reduce the sulfur of CF₃S(═O)ONa in the course of the sulfur-fonctionalization of the pyrazole ring, thus leading to the formation of the sulfide compound of formula I.

This was quite unexpected, as a wide variety of chlorinating agents have been reported to effect sulfinylation of the pyrazole ring in similar reaction conditions. For example, EP 0 668 269 [ref 2] describes a one step sulfinylation process involving the reaction of a reagent RS(═O)X with the heterocycle to afford the desired sulfinylated compound. According to EP 0 668 269, typical chlorinating agents such as phosgene, chloroformates, PCl₅ and SOCl₂ can effect direct sulfinylation of the pyrazole ring in conjunction of a reagent RSOX, depending on the nature of X. In that same document, direct sulfinylation was also described with the use of CF₃SO₂H or CF₃SO₂Na in conjunction with a chlorinating agent such as SOCl₂ or phosgene. Similarly, Chinese patent N° CN 1176078C [ref 1] describes a sulfinylation process using a mixture of CF₃SO₂K and CF₃SO₂Na in the presence of a chlorinating agent such as POCl₃, PCl₃ or SOCl₂. Neither one of these two documents reported the possibility of accessing the sulfide with the combination of a chlorinating agent and a reagent such as CF₃S(═O)ONa. In fact, both of these processes were described as having the advantage of avoiding the formation of such sulfide and the need for a subsequent oxidation step to yield the desired sulfoxide (e.g., fipronil).

As used herein, the term “selective oxidizing agent” refers to an oxidizing agent that effects oxidation of a thioether selectively to the corresponding sulfoxide, while minimizing the formation of the sulfone. More specifically, the “selective oxidizing agent” according to the invention effects oxidation of thioether (I) or (IA) selectively to the corresponding sulfoxide (II) or (IIA), respectively. The term “selectively”, as used in this context, means that the desired sulfoxide (II) (or (IIA)) is formed predominantly over the corresponding sulfone. In certain embodiments, step b) of the inventive process leads to the formation of sulfoxide (II) and its corresponding sulfone (IV) (or sulfoxide (IIA) and its corresponding sulfone (IVA)) in a ratio sulfoxide:sulfone ≧50:50, for example ≧55:45, for example ≧60:40, for example ≧65:35, for example ≧70:30, for example ≧75:25, for example ≧80:20, for example ≧85:15, for example ≧90:10, for example ≧95:5, for example ≧96:4, for example ≧97:3, for example ≧98:2, for example ≧99:1, for example 100:0.

Control of the selectivity may be due to the nature of the oxidizing agent itself, or to the reaction conditions in which it is employed, or both.

Such selective oxidizing agents, and suitable reaction conditions, to effect selective oxidation of thioethers to the corresponding sulfoxide are known in the art.

For example, it has been reported that meta-chloroperbenzoic acid (“MCPBA”) among the oxidants can selectively oxidize a sulfide compound to the corresponding sulfoxide when used in an equivalent amount at low temperature (usually, −78° C. to 0° C.) in the presence of dichloromethane solvent, while selectively oxidize a sulfide to the corresponding sulfone when used in an amount of two equivalents at room temperature (Nicolaou, K. C.; Magolda, R. L.; Sipio, W. J.; Barnette, W. E.; Lysenko, Z.; Joullie, M. M., J. Am. Chem. Soc. 1980. 102, 3784; [ref 5]).

In practice, MCPBA is typically employed in an excess amount, since the accurate amount cannot be evaluated as it is commercially merchandised in 60-80% purity. MCPBA is also relatively expensive, and involves the problem of treating meta-chlorobenzoic acid as by-product. It is thus seldom used in processes on an industrial scale. Nevertheless, MCPBA can be used for carrying out the process of the process (on labscale for example), and is thus considered to fall within the scope of the invention.

Other selective oxidating agents have been reported. For example, the following recent publications may be mentioned:
1. Khodaei et al., <<H₂O₂/Tf₂O System: An Efficient Oxidizing Reagent for Selective Oxidation of Sulfanes>>, Synthesis 2008 (11) 1682 [ref 6];
2. Y. Venkateswarlu et al., <<A novel rapid sulfoxidation of sulfides with cyclohexylidenebishydroperoxide>> Tetrahedron Letters 2008 (49) 3463 [ref 7];
3. Ali et al., <<Ceric Ammonium Nitrate Catalyzed Oxidation of Sulfides to Sulfoxides>>, Synthesis 2007 (22) 3507 [ref 8];
4. Yu Yuan, Yubo Bian, <<Gold(III) catalyzed oxidation of sulfides to sulfoxides with hydrogen peroxide>> Tetrahedron Letters 2007 (48) 8518 [ref 9];
5. S. B. Halligudi et al., <<One-step synthesis of SBA-15 containing tungsten oxide nanoclusters: a chemoselective catalyst for oxidation of sulfides to sulfoxides under ambient conditions>> Chem. Commun. 2007 4806 [ref 10].

The above publications all report a high selectivity towards mono-oxidation to the sulfoxide. As such, the oxidation methods described therein may be applied to step b) of the process of the invention, with reasonably good expectation of high selectivity towards the desired sulfoxide (II) or (IIA).

Exemplary reduction to practice of these methods are illustrated in Examples 9 through 12 below. It is understood that the procedures exemplified in the Examples can be modified and adjusted by the skilled artisan in order to define optimal conditions for obtaining Fipronil (II), or more generally compounds of formula (IIA), in good yields and high purity.

The oxidizing agents described in the above publications, and in Examples 9 through 12 below, fall within the scope of the invention. However, the selective oxidizing agents suitable for use in the process of the invention are not limited to these examples. It is understood that any oxidizing agent or conditions that lead to selective oxidation of thioether (I) or (IA) to the corresponding sulfoxide (II) or (IIa), respectively, is considered to fall within the scope of the invention.

For example, another important aspect of the present invention is the recognition that MHSO₅, in particular oxone (KHSO₅), is an effective oxidizing agent that enables the controlled oxidation of the sulfide of formula I to the sulfoxide of formula II (fipronil), without excessive formation of the corresponding sulfone. As the person of ordinary skill in the art will appreciate, one difficulty to overcome is to identify an oxidizing agent having a “balanced” oxidizing power. On the one hand, the oxidizing agent should be sufficiently reactive to enable the oxidation of electron deficient sulfides such as trifluoromethylsulfides, which are less readily oxidized than other sulfides. On the other hand, the oxidizing agent should not so potent that an excessive formation of the undesired sulfone will occur. The inventors have recognized that the reagent MHSO₅ had the adequate chemical properties to serve this purpose. They also developed and designed proper oxidation reaction conditions that enable the selective formation of fipronil over the undesired sulfone of formula IV.

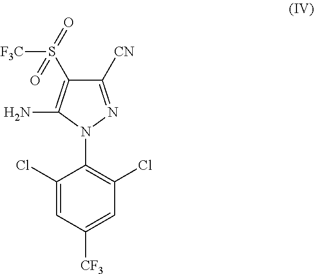

### Embodiments Relating to the First Aspect of the Invention, and Step a) of the Third Aspect of the Invention

In certain embodiments, at least one equivalent of the reducing/halogenating agent is used, based on the molar amount of CF₃S(═O)ONa. In certain exemplary embodiments, the reducing/halogenating agent (RHA) and CF₃S(═O)ONa are used in a molar ratio RHA/CF₃S(═O)ONa ranging from 1.0 to 2.0, preferably from 1.0 to 1.7, more preferably from 1.0 to 1.5, most preferably from 1.0 to 1.3. In certain exemplary embodiments, the reducing/halogenating agent is PCl₃ or PBr₃. In certain preferred embodiments, the reducing/halogenating agent is PCl₃.

In certain embodiments, a reagent having the structure RSO₂Na can be used in place of CF₃SO₂Na, wherein R is a C₁₋₄haloalkyl. Thus, the present invention provides a process for preparing compounds of formulae IA and IIA:

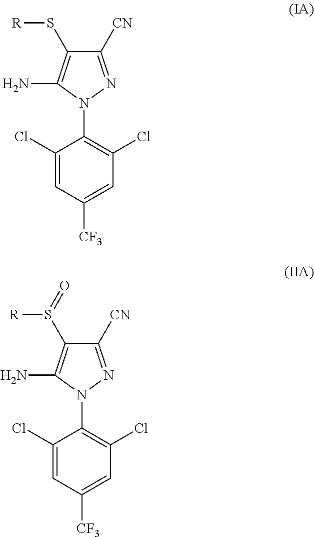

In certain embodiments, step b) of the process of the invention is carried such that little or no formation of sulfone (IVA) occurs.

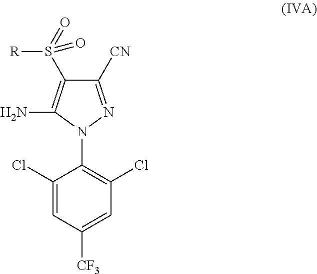

In certain exemplary embodiments, R represents a C₁₋₃haloalkyl group. In certain exemplary embodiments, R represents a C₁₋₂haloalkyl group. In certain exemplary embodiments, R is a halomethyl group. In certain other exemplary embodiments, R is CF₃.

In certain embodiments, the process is carried out in the presence of an amine salt, the amine being a primary, secondary or tertiary amine. For example, the amine salt may be a methylamine, ethylamine, propylamine, isopropylamine, pyridine, dimethylamine, diethylamine, trimethylamine or triethylamine salt. In certain embodiments, the amine salt is a hydrochloride salt. In certain embodiments, the amine salt is a sulfonic acid salt. In certain exemplary embodiments, the amine salt is a methyl sulfonic acid (mesylate), benzene sulfonic acid or para-toluene sulfonic acid salt (PTSA, tosylate salt). In certain exemplary embodiments, the process is carried out in the presence of dimethylamine tosylate salt (NHMe₂.PTSA).

In certain embodiments, the molar ratio between the amine salt and the compound of formula III is <1 (the amine salt is used in catalytic amounts). In certain exemplary embodiments, the molar ratio between the amine salt and the compound of formula III is between 1.0 and 2.0, preferably between 1.0 and 1.9, more preferably between 1.0 and 1.8, more preferably between 1.0 and 1.7, more preferably between 1.0 and 1.6, most preferably between 1.0 and 1.5.

The process may be carried out in a variety of solvents, or mixture of solvents. Any solvent or mixture of solvents that allows the reaction of the different reagents and/or compounds involved may be used. For example, the solvent may be selected from diethyl ether, dichloromethane, 1,2-dichloroethane, tetrahydrofuran (THF), 2-methyl-tetrahydrofuran (MeTHF), dimethyl formamide (DMF), toluene, benzene, dimethyl sulfoxide (DMSO), or a combination of two or more of them. In other embodiments, the solvent may be selected from n-heptane, cyclohexane, benzene, xylene, tert-butyl methyl ether (TBME), DMF, THF, chloroform, ethyl acetate, dichloromethane, 1,2-dichloroethane, 2-methyltetrahydrofuran, acetonitrile or CCl₄), or a combination of two or more of them. A mixture of solvents may be used, and the solvents may differ in polarity. For example, a mixture of toluene and DMF may be used.

In certain embodiments, the progress of the reaction may be monitored, for example by spectroscopic means (e.g., ¹H NMR, ¹³C NMR and/or LCMS) and/or chromatographic means (e.g., HPLC and/or TLC). For example, reaction mixture aliquots may be sampled at intervals throughout the reaction and analyzed to determine the conversion ratio [compound of formula III]/[compound of formula I].

### Embodiments Relating to the Second Aspect of the Invention, and Step b) of the Third Aspect of the Invention

Any oxidizing agent or conditions that lead to selective oxidation of thioether (I) or (IA) to the corresponding sulfoxide (II) or (IIa), respectively, may be used to selectively oxidize thioether (I) (or (IA)) to the corresponding sulfoxide.

In certain embodiments, the selective oxidizing agent may be H₂O₂/Tf₂O. The skilled practitioner can adapt the method and reaction conditions described in ref 6 to carry out step b) of the process of the invention. An exemplary (but not limitative) methodology is described in Example 9 below.

In certain other embodiments, the selective oxidizing agent may be cyclohexylidenebshydroperoxide. The skilled practitioner can adapt the method and reaction conditions described in ref 7 to carry out step b) of the process of the invention. An exemplary (but not limitative) methodology is described in Example 10 below.

In certain other embodiments, the selective oxidizing agent may be Ceric ammonium nitrate (CAN) and sodium bromate (NaBrO₃). The skilled practitioner can adapt the method and reaction conditions described in ref 8 to carry out step b) of the process of the invention. An exemplary (but not limitative) methodology is described in Example 11 below.

In certain other embodiments, the selective oxidizing agent may be H₂O₂ in the presence of hydrogen tetrachloroaurate(III) hydrate. The skilled practitioner can adapt the method and reaction conditions described in ref 9 to carry out step b) of the process of the invention. An exemplary (but not limitative) methodology is described in Example 12 below.

In certain other embodiments, the selective oxidizing agent may be MHSO₅ under suitable conditions, wherein M is an alkaline metal cation.

Paragraphs [0051] through [0064] relate to embodiments in which the selective oxidizing agent is MHSO₅ wherein M is an alkaline metal cation.

As the skilled artisan will appreciate, the step of oxidizing the compound of formula I in the presence of KHSO₅ can lead to the formation of the corresponding sulfone (of formula IV) if the reaction conditions are favorable.

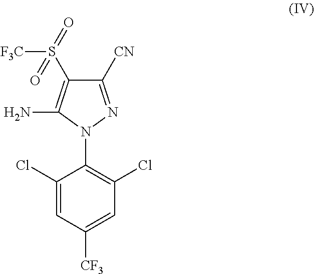

Nevertheless, careful control of the reaction conditions allows the selective formation of the desired sulfinylated compound of formula II (fipronil). For example, the control of one or more parameters such as the amount of MHSO₅ used, the reaction temperature, the addition rate of oxone, the reaction time and/or the solvent system can help direct the oxidation reaction toward the selective formation of compound of formula II over the corresponding sulfone of formula IV

The amount of MHSO₅ influences the oxidation reaction since an excess will lead to the formation of the corresponding sulfone (compound of formula IV), while a deficiency will lead to incomplete transformation, and in either event an impure final product is obtained. Accordingly, proper care is given to the molar amount of MHSO₅ that is used to carry out this reaction step. In certain embodiments, the compound of formula I and MHSO₅ are used in a molar ratio compound I/MHSO₅ ranging from 1.0 to 2.0, preferably from 1.0 to 1.8, more preferably from 1.0 to 1.6, most preferably from 1.0 to 1.4. In certain exemplary embodiments, MHSO₅ is KHSO₅ (oxone).

In certain embodiments, selective formation of fipronil over the corresponding sulfone of formula IV is effected, in whole or in part, by controlling the reaction temperature. Thus, in certain embodiments, the oxidation reaction is carried out at a temperature ranging from −20° C. to −10° C., preferably from −15° C. to −10° C. In certain exemplary embodiments, the oxidation reaction is carried out at a temperature ranging from −20° C. to −5° C. In certain exemplary embodiments, the oxidation reaction is carried out at −15° C.±3° C.

In certain embodiments, selective formation of fipronil over the corresponding sulfone of formula IV is effected, in whole or in part, by controlling the addition rate of MHSO₅ to the reaction mixture comprising the compound of formula I. Thus, in certain embodiments, in the step of oxidizing the compound of formula I, MHSO₅ is added portionwise. In certain exemplary embodiments, MHSO₅ is added by portions while the reaction temperature is maintained between −20° C. to −10° C., more preferably −15° C. to −10° C., most preferably about −10° C. In certain exemplary embodiments, MHSO₅ is KHSO₅ and the addition of KHSO₅ is done portionwise while maintaing the reaction temperature at about −10° C.

In certain embodiments, selective formation of fipronil over the corresponding sulfone of formula IV is effected, in whole or in part, by controlling the solvent system used to carry out the oxidation step b).

For example, in certain exemplary embodiments, the solvent comprises an organic acid, such as trifluoroacetic acid (TFA) or acetic acid. In certain exemplary embodiments, the organic acid is trifluoroacetic acid (TFA). In certain exemplary embodiments, when TFA is used as the solvent, or as part of the solvent system, MHSO₅ is added by portions while the reaction temperature is maintained between −20° C. to −10° C., more preferably −15° C. to −10° C., most preferably about −10° C. In certain exemplary embodiments, MHSO₅ is KHSO₅ and the addition of KHSO₅ is done portionwise while maintaining the reaction temperature at about −10° C. Reaction time may be optimized experimentally. In certain exemplary embodiments, when TFA is used as the solvent, or as part of the solvent system, the oxidation step b) can be carried for a time period ranging from 6 to 12 hours, more preferably from 8 to 12 hours, most preferably about 8 hours, for example at the temperature ranges given above.

In other embodiments, the solvent comprises a halogenated alcohol, such as tetrafluoropropanol (TFP). In certain exemplary embodiments, the solvent is TFP. In general, when TFP is used as the solvent, or as part of the solvent system, the oxidation step b) can be carried out between 25 and 55° C., more preferably between 25 and 45° C., most preferably between 25 and 30° C. Reaction time may be optimized experimentally. In certain exemplary embodiments, when TFP is used as the solvent, or as part of the solvent system, MHSO₅ may be added by portions and the oxidation step b) can be carried for 24 to 72 hours, more preferably for 24 to 48 hours, for example at the temperature ranges given above. The reaction conditions (e.g., temperature of addition of oxone, reaction time and/or temperature) may be optimized experimentally.

In certain embodiments, selective formation of fipronil over the corresponding sulfone of formula IV is effected, in whole or in part, by controlling the oxidation reaction time (i.e., the time that MHSO₅ (e.g., oxone, or KHSO₅) is allowed to react with the compound of formula I). Thus, in certain embodiments, when the oxidizing reaction is conducted at about −15° C., in the step of oxidizing the compound of formula I, MHSO₅ is allowed to react with the compound of formula I for a time period ranging from 6 to 12 hours, more preferably from 8 to 12 hours, most preferably about 8 hours. In certain exemplary embodiments, MHSO₅ is KHSO₅ and the oxidizing reaction is carried out at about −15° C. for about 8 hours.

In certain embodiments, selective formation of fipronil over the corresponding sulfone of formula IV is effected, in whole or in part, by controlling (i) the amount of MHSO₅ used, (ii) the reaction temperature, (iii) the addition rate of MHSO₅ to the reaction mixture comprising the compound of formula I, and (iv) the oxidation reaction time (i.e., the time that MHSO₅) is allowed to react with the compound of formula I).

Thus, in certain embodiments, in the step of oxidizing the compound of formula I, an organic acid such as TFA is used as the solvent, or as part of the solvent system, and:
(i) the compound of formula I and MHSO₅ are used in a molar ratio compound I/MHSO₅ ranging from 1.0 to 2.0, preferably from 1.0 to 1.8, more preferably from 1.0 to 1.6, most preferably from 1.0 to 1.4. In certain exemplary embodiments, MHSO₅ is KHSO₅ (oxone);
(ii) the oxidation reaction is carried out at a temperature ranging from −20° C. to −10° C., preferably from −15° C. to −10° C., most preferably at about −15° C.;
(iii) MHSO₅ is added by portions while the reaction temperature is maintained between −20° C. to −10° C., more preferably −15° C. to −10° C., most preferably about −10° C.; and
(iv) MHSO₅ is allowed to react with the compound of formula I for a time period ranging from 6 to 12 hours, more preferably from 8 to 12 hours, most preferably about 8 hours. In certain exemplary embodiments, MHSO₅ is KHSO₅ and the oxidizing reaction is carried out at about −15° C. for about 8 hours.

In certain embodiments, the step of oxidizing is carried out in the presence of an organic acid, such as trifluoroacetic acid (TFA) or acetic acid. In certain exemplary embodiments, the organic acid is trifluoroacetic acid (TFA).

In certain embodiments, the organic acid is used in large excess (>10 equivalents), based on the molar amount of MHSO₅. In certain exemplary embodiments, the organic acid id TFA.

In certain other embodiments, in the step of oxidizing the compound of formula I, an halogenated alcohol such as TFP is used as the solvent, or as part of the solvent system, and:
(i) the compound of formula I and MHSO₅ are used in a molar ratio compound I/MHSO₅ ranging from 1.0 to 2.0, preferably from 1.0 to 1.8, more preferably from 1.0 to 1.6, most preferably from 1.0 to 1.4. In certain exemplary embodiments, MHSO₅ is KHSO₅ (oxone);
(ii) the oxidation reaction is carried out at a temperature ranging between 25 and 55° C., more preferably between 25 and 45° C., most preferably between 25 and 30° C.;
(iii) MHSO₅ is added by portions while the reaction temperature is maintained between . . . ° C. to . . . ° C., more preferably . . . ° C. to . . . ° C., most preferably about . . . ° C.; and
(iv) MHSO₅ is allowed to react with the compound of formula I for a time period ranging from 24 to 72 hours, more preferably for 24 to 48 hours. In certain exemplary embodiments, MHSO₅ is KHSO₅ and the oxidizing reaction is carried out at about 27-30° C. for about 48 hours.

In general, as applied to all the above embodiments regarding the selective oxidizing agent, step b) of the process may be carried out in a variety of solvents, or mixture of solvents. Any solvent or mixture of solvents that allows the reaction of the different reagents and/or compounds involved may be used. For example, the solvent may be selected from diethyl ether, dichloromethane, 1,2-dichloroethane, tetrahydrofuran (THF), 2-methyl-tetrahydrofuran (MeTHF), dimethyl formamide (DMF), toluene, benzene, dimethyl sulfoxide (DMSO), or a combination of two or more of them. In other embodiments, the solvent may be selected from n-heptane, cyclohexane, benzene, xylene, tert-butyl methyl ether (TBME), DMF, THF, chloroform, ethyl acetate, dichloromethane, 1,2-dichloroethane, 2-methyltetrahydrofuran, acetonitrile or CCl₄), or a combination of two or more of them. A mixture of solvents may be used, and the solvents may differ in polarity. In certain embodiment, an organic acid such as TFA is used as the solvent.

In certain embodiments, as applied to all the above embodiments regarding the selective oxidizing agent, the progress of the oxidation reaction may be monitored, for example by spectroscopic means (e.g., ¹H NMR, ¹³C NMR and/or LCMS) and/or chromatographic means (e.g., HPLC and/or TLC). For example, reaction mixture aliquots may be sampled at intervals throughout the reaction and analyzed to determine the conversion ratio [compound of formula I]/[compound of formula II] and/or to monitor the presence/formation of the undesired sulfone of formula IV.

In certain embodiments, as applied to all the above embodiments regarding the selective oxidizing agent, fipronil (product of formula II) obtained by the inventive process may be recrystallized in a suitable solvent. For example, fipronil may be recrystallized from a suitable solvent system such as toluene, ethylacetate, isopropyl acetate, or a combination of two or more of them. In certain exemplary embodiments, fipronil is recrystallized from toluene.

In certain embodiments, as applied to all the above embodiments regarding the selective oxidizing agent, the process of the invention allows the preparation of fipronil with a purity >95.0%, more preferably ≧95.1%, still more preferably ≧95.3%, still more preferably ≧95.5%, still more preferably ≧95.7%, still more preferably ≧95.9%, still more preferably ≧96.0%, still more preferably ≧96.5%, still more preferably ≧97.0%, still more preferably ≧97.5%, still more preferably ≧98.0%, still more preferably ≧98.5%, still more preferably ≧99.0%, still more preferably ≧99.1%, still more preferably ≧99.2%, still more preferably ≧99.3%, still more preferably ≧99.4%, still more preferably ≧99.5%, still more preferably ≧99.6%, still more preferably ≧99.7%, still more preferably ≧99.8%, still more preferably ≧99.9%. In certain exemplary embodiments, fipronil obtainable by the inventive process has a purity ranging from 97 and 98%. In certain embodiments, the purity is assessed by HPLC.

In another aspect, as applied to all the above embodiments regarding the selective oxidizing agent, there is provided a compound of formula IIA obtainable by the process of the invention. In certain embodiments, the compound of formula IIA obtainable by the process of the invention has a purity >95.0%, more preferably ≧95.1%, still more preferably ≧95.3%, still more preferably ≧95.5%, still more preferably ≧95.7%, still more preferably ≧95.9%, still more preferably ≧96.0%, still more preferably ≧96.5%, still more preferably ≧97.0%, still more preferably ≧97.5%, still more preferably ≧98.0%, still more preferably ≧98.5%, still more preferably ≧99.0%, still more preferably ≧99.1%, still more preferably ≧99.2%, still more preferably ≧99.3%, still more preferably ≧99.4%, still more preferably ≧99.5%, still more preferably ≧99.6%, still more preferably ≧99.7%, still more preferably ≧99.8%, still more preferably ≧99.9%. In certain exemplary embodiments, the compound of formula IIA obtainable by the process of the invention has a purity ranging from 97 and 98%. In certain embodiments, the purity is assessed by HPLC.

In a fourth aspect, there is provided the use of fipronil obtainable by the process described herein for the preparation of an antiparasitic composition for therapeutic use.

In a fifth aspect, there is provided the use of the process described herein for the preparation of an antiparasitic composition for therapeutic use. In particular, there is provided a process for manufacturing an antiparasitic medicament comprising carrying out the process as described in the various embodiments of the third aspect of this invention, and mixing the fipronil obtained by said process with a pharmaceutically acceptable carrier, adjuvant or vehicle.

In certain embodiments of the fourth and the fifth aspects above, the antiparasitic composition is used for veterinary applications. In certain embodiments, the antiparasitic composition is used for treating domestic animals such as cats and dogs. In certain exemplary embodiments, the fipronil obtainable by the inventive process is used as an antiparasitic agent for preventing or eradicating pests such as fleas and ticks in domestic animals such as cats and dogs.

The inventive process has several advantages over known processes.

First, it allows to gain technically easier access to the thioether intermediate of formula I. Known processes for the preparation of this thioether typically involve using gaseous, volatile, expensive and unstable trifluoromethylsulfenylchloride (CF₃SC1). In contrast, the present process uses reagents that are technically safer, and that do not require the use of pressure equipment for the containment of gases.

Second, the possibility of conveniently accessing the thioether intermediate of formula I with an overoxidation of <3.5%, preferably <2.5% is an advantage in and of itself. In particular, we note that sulfoxides are generally more reactive, more prone to be oxidized to the compound of formula IV—which is not desirable (<3.5%, preferably <2.5%). Accordingly, the present process can be viewed as allowing the storage of fipronil in the more stable sulfide form. Thus, the inventive process presents an economical advantage in that massive amounts of fipronil can be prepared with limited losses (due to the product decomposition), since fipronil can be prepared and stored in its more stable sulfide form before the final oxidation step is carried out.

Third, the present process enables the preparation of fipronil in high purity (e.g., ≧96%). It is thus particularly adapted for the synthesis of this antiparasitic agent for therapeutic use, as opposed to agricultural and/or horticultural use, for which the purity level is not as crucial.

Finally, the inventive process allows the preparation of fipronil in good yields.

In summary, the present process has all the essential features that a viable and efficient industrial process requires. As such, unlike other known processes in the art, it is particular adapted for the mass production of <<therapeutical grade>> fipronil (i.e., sufficiently pure fipronil that it is suitable for therapeutic use).

As discussed above, the present invention provides compositions comprising fipronil obtainable by the process of the invention for use as an antiparasitic medicament. Accordingly, in another aspect of the present invention, pharmaceutically acceptable compositions are provided, wherein these compositions comprise fipronil obtainable by the process of the invention as described herein, and optionally comprise a pharmaceutically acceptable carrier, adjuvant or vehicle. In certain embodiments, these compositions optionally further comprise one or more additional therapeutic agents.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980 [ref 11]) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Dosage forms for topical or transdermal administration of a composition of this invention include ointments, pastes, creams, lotions, gels, powders, solutions, sprays, inhalants or patches. The active component (fipronil) is generally admixed under sterile conditions with a pharmaceutically acceptable carrier and any needed preservatives or buffers as may be required.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of liquid topical forms. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Optionally associated with such container(s) can be a notice in the form prescribed by a governmental agency regulating the manufacture, use or sale of pharmaceutical products, which notice reflects approval by the agency of manufacture, use or sale for animal administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

The process of this invention and its modes of reduction to practice can be understood further by the examples that follow. It will be appreciated, however, that these examples do not limit the invention. Variations of the invention, now known or further developed, are considered to fall within the scope of the present invention as described herein and as hereinafter claimed.

### Example 1

### Industrial Scale Purification of CF₃SO₂Na

In a 500 L reactor, 75.0 kg of commercially available CF₃SO₂Na was added, followed by 210 kg of ethyl acetate. The resulting mixture was stirred at 25±5° C. for 1 hour. Silicon gel (10.7 kg) was added. The resulting mixture was stirred for 15 minutes, and then filtered by centrifugation. The filter cake (residue) was added to a 200 L reactor and 76.3 kg of ethyl acetate was added. The resulting mixture was stirred at 25±5° C. for 1 hour, and was then filtered by centrifugation. The filter cake (residue) was reintroduced into the reactor and the procedure (ethyl acetate and filtration) was repeated one more time using 76.3 kg of ethyl acetate. The washing process was repeated 2 to 3 times

The filtrates were combined and 106.6 kg of pure deionized water was added. The resulting mixture was heated to 50±5° C. and was stirred at that temperature for 30 minutes and then cooled to room temperature. The organic layer was separated and 106.6 kg of water was added. The resulting mixture was heated to 50±5° C., was stirred at that temperature for 30 minutes, and was then cooled to 20±5° C. The aqueous and organic layers were separated. The combined aqueous layers were extracted once with 73.5 kg of CH₂Cl₂ in three portions. The organic layer was concentrated under reduced pressure at 70° C. Toluene (100.0 kg) was added to the residue; The resulting mixture was distilled and the residual water separated out under vacuum at 70° C. 84.0 kg of toluene was added to the residue. CF₃SO₂Na was stored as a solution in toluene.

### Example 2

### Industrial Scale Preparation of Catalyst PTSA-NHMe₂

In a 200 L reactor, 70.0 kg of PTSA was added. Me₂NH (5805 g, 30% aq. Solution) was added dropwise at 25±5° C. The resulting solution was stirred at that temperature for 1 hour. The solution was then concentrated under vacuum at 70±5° C. Toluene (100.0 kg) was added to the residue. Residual water was removed by azeotropic distillation under vacuum at 70±5° C. When no more water could be separated out, the mixture was cooled to 20±5° C., and filtered over a 1.0 mm porous titanium alloy filtration cartridge with pressure nitrogen purge. The filter cake was dried under vacuum at 70±5° C.

### Example 3

### Industrial Scale Preparation of Compound of Formula I

In a 200 L reactor, 12.0 kg of 5-amino-1-(2,6-dichloro-4-(trifluoromethyl)phenyl)-1H-pyrazole-3-carbonitrile (compound of formula III), 11.7 kg of CF₃SO₂Na obtained in Example 1, 12.4 kg of catalyst PTSA.NHMe₂ obtained in Example 2, and 90.8 kg of toluene were added. The resulting mixture was stirred at room temperature (25+/−5° C.) for 15 minutes, and 0.11 kg of DMF was added. The resulting mixture was stirred at room temperature for 30 minutes. The mixture was cooled to 0±2° C., and PCl₃ (5.1 g) was added dropwise at that temperature. The resulting mixture was stirred at 0±2° C. for 1 hour. It was then warmed to room temperature and stirred for 1 hour at 20±5° C. The mixture was then heated to ˜65-70° C., and was stirred at that temperature for 8 hours.

Water (48.0 kg) and 16.1 kg of ethyl acetate were added. The resulting mixture was stirred for 30 minutes, cooled at room temperature and separated. The organic layer was concentrated under vacuum at 65° C. Toluene (31.1 kg) was added to the residue. The resulting mixture was heated to 90±5° C., then slowly cooled to ˜10-15° C., and stirred for 2 hours at that temperature. The mixture was filtered, and the filter cake was dried under vacuum at 60±2° C. If the purity of the crude product was <96%, it was recrystallized from toluene.

### Example 4

### Industrial Scale Preparation of Compound of Formula II

In a 100 L reactor, 10.0 kg of the crude product (or recrystallized product) obtained in Example 3 and 74.0 kg of TFA were added The resulting mixture was stirred for 15 minutes, and was then cooled to −15° C. Oxone (13.9 kg) was added portionwise at −15±5° C. The resulting mixture was stirred at that temperature until the amount of starting material (compound of formula I) in the reaction mixture was ≦1.5% or until the amount of corresponding sulfone (compound of formula IV) detected in the reaction mixture was ≧2%. The reaction mixture was then poured into a cool (−20 to −10° C.) solution of 12.0 kg of Na₂SO₃ in 220 kg of deionized water. The resulting mixture was stirred for 30 minutes, and as then filtered. The presence of peroxide was checked with KI+ starch test paper. Ethyl acetate (44.8 kg) and 30.0 kg of water were added to the filter cake. The resulting mixture was stirred for 30 minutes. The pH of the mixture was then adjusted to ˜8-9 with a saturated aqueous solution of Na₂CO₃. The aqueous layer was separated and was extracted once with 26.9 kg of ethyl acetate. The combined organic layers were washed with 40.0 kg of brine. The organic layer was separated, and was concentrated under vacuum at 50° C. CH₂Cl₂ (40.0 kg) was added to the residue. The mixture was stirred at 35±5° C. for 3 hours. It was then cooled to 10±5° C., was stirred for 2 hours, and was then filtered. Toluene (73.5 kg) was added to the filter cake. The resulting mixture was heated to reflux (˜105° C.), filtered, then slowly cooled to ˜10-15° C., and stirred for 2 hours at that temperature. The mixture was filtered, and the filter cake was dried under vacuum at 60±5° C. If the purity of the crude product was <96%, it was recrystallized in toluene to raise the purity>96%. A 50% overall yield was obtained.

### Example 5

### Laboratory Scale Purification of CF₃SO₂Na

In a 10 L four-necked flask equipped with a thermometer and mechanical stirrer, 1.759 kg of commercially available CF₃SO₂Na was added, followed by 5.50 L of ethyl acetate. The resulting mixture was stirred at 20±5° C. for 1 hour. Silicon gel (250 g) was added. The resulting mixture was stirred for 15 minutes, and was then filtered. The filter cake (residue) was added to the flask and 2.0 L of ethyl acetate was added. The resulting mixture was stirred at 20±5° C. for 1 hour, and was then filtered. 2.50 L of water was added to the combined filtrates. The resulting mixture was heated to 50±5° C. and was stirred at that temperature for 30 minutes and then cooled to 20±5° C. The organic layer was separated and 2.50 L of water was added. The resulting mixture was heated to 50±5° C., was stirred at that temperature for 30 minutes, and was then cooled to 20±5° C. The aqueous and organic layers were separated. The combined aqueous layers were extracted with 1.30 L of CH₂Cl₂. The organic layer was concentrated under reduced pressure at 72° C. Toluene (1.00 L) was added to the residue. The resulting mixture was azeotropically distilled under vacuum at 72° C. to give 767.7 g CF₃SO₂Na (72.7%).

### Example 6

### Laboratory Scale Preparation of Catalyst PTSA-NHMe₂

In a 2 L four-necked flask equipped with a thermometer, a drop funnel and a mechanical stirrer, 500.0 g of PTSA was added. Me₂NH (418.0 g, 30% aq. Solution) was added dropwise at 25±5° C. The resulting solution was stirred at that temperature for 1 hour. The solution was then concentrated under vacuum at 70±5° C. Toluene (300.0 mL) was added to the residue. Residual water was removed by azeotropic distillation under vacuum at 70±5° C. The distillation was repeated with 160.0 mL of toluene. 160 mL of isopropyl alcohol (IPA) was added to the residue. The resulting mixture was heated to 90° C. and was stirred at that temperature (90±5° C.) for 1.5 hours. After cooling to 4° C., the mixture was filtered. The filter cake was dried under vacuum at 65±5° C. to give 561.1 g of desired product (98.3% yield).

### Example 7

### Laboratory Scale Preparation of Compound of Formula I

In a 3 L four-necked flask equipped with a thermometer, a drop funnel and a mechanical stirrer, 200 g of 5-amino-1-(2,6-dichloro-4-(trifluoromethyl)phenyl)-1H-pyrazole-3-carbonitrile (compound of formula III), 194.4 g of CF₃SO₂Na obtained in Example 5, 206.2 g of catalyst PTSA.NHMe₂ obtained in Example 6, and 1750 mL of toluene were added. The resulting mixture was stirred at room temperature (25+/−5° C.) for 15 minutes, and 2.00 mL of DMF was added. The resulting mixture was stirred at room temperature for 30 minutes. The mixture was cooled to 0±2° C., and PCl₃ (85.0 g) was added dropwise at that temperature. The resulting mixture was stirred at 0±2° C. for 1 hour. It was then warmed to room temperature and stirred for 1 hour at 20±5° C. The mixture was then heated to 70° C.±5° C., and was stirred at that temperature for 6 hours.

Water (800 mL) and 300 mL of ethyl acetate were added. The resulting mixture was stirred for 30 minutes, cooled at room temperature and separated. The organic layer was concentrated under vacuum at 50° C. to give 350.7 g of residue. Toluene (600 mL) was added to the residue. The resulting mixture was heated to 90±5° C., then slowly cooled to ˜10-15° C., and stirred for 2 hours at that temperature. The mixture was filtered, and the filter cake was dried under vacuum at 60±2° C. to give 181.7 g of desired product (66.7% yield; 97.7% pure).

The reaction was also conducted in a variety of other solvents in good yields. For example, the thioether (I) can be prepared from 5-amino-1-(2,6-dichloro-4-(trifluoromethyl)phenyl)-1H-pyrazole-3-carbonitrile (compound of formula III) using the experimental protocol described above, wherein DMF is replaced with n-heptane, cyclohexane, benzene, xylene, tert-butyl methyl ether (TBME), THF, chloroform, ethyl acetate, dichloromethane, 1,2-dichloroethane, 2-methyltetrahydrofuran, acetonitrile or CCl₄.

### Example 8

### Laboratory Scale Preparation of Compound of Formula II Using Oxone as Oxidizing Agent

In a 1 L four-necked flask equipped with a thermometer and a mechanical stirrer, 100 g of the crude product obtained in Example 7 and 700 mL of TFA were added. The resulting mixture was stirred for 15 minutes, and was then cooled to −15° C. Oxone (139.3 g) was added portionwise at −15±5° C. The resulting mixture was stirred at that temperature until the amount of starting material (compound of formula I) in the reaction mixture was ≦1.5% or until the amount of corresponding sulfone (compound of formula IV) detected in the reaction mixture was ≧2%. The reaction mixture was then poured into a cool (−20 to −10° C.) solution of 120 g of Na₂SO₃ in 2200 g of water. The resulting mixture was stirred for 30 minutes, and was then filtered. Ethyl acetate (500 mL) and 300 mL of water were added to the filter cake. The resulting mixture was stirred for 30 minutes. The pH of the mixture was then adjusted to 8 with a saturated aqueous solution of Na₂CO₃. The aqueous layer was separated and was extracted once with 300 mL of ethyl acetate. The combined organic layers were washed with 400 mL of brine. The organic layer was separated, and was concentrated under vacuum at 50° C. Toluene (850 mL) was added to the residue. The resulting mixture was heated to reflux (˜105° C.), filtered, then slowly cooled to ˜10-15° C., and stirred for 2 hours at that temperature. The mixture was filtered, and the filter cake was dried under vacuum at 60±2° C. CH₂Cl₂ (200 mL) was added to the product. The mixture was stirred at 25-35° C. for 2 hours, and then was filtered. CH₂Cl₂ (300 mL) was added to the product. The mixture was stirred at 25-35° C. for 1 hour, and then was filtered. CH₂Cl₂ (250 mL) was added to the product. The mixture was stirred at 25-35° C. for 5 hours, and then was filtered and dried under vacuum at 50° C. to give 56.8 g of desired product (55.4% yield; 97.1% pure).

### Example 9

### Laboratory Scale Preparation of Compound of Formula II Using H₂O₂/Tf₂O as Oxidizing Agent

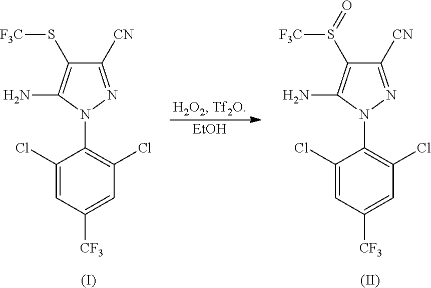

In a 0.5 liter 3-necked round bottom flask equipped with a dropping funnel, a reflux condenser, a mechanical stirrer, a thermometer and an inert gas supply, 16.84 g thioether (I) (40 mmol) was dissolved under nitrogen in 200 ml ethanol and treated with 8.0 ml 30% aqueous hydrogenperoxide (80 mmol) and 3.3 ml trifluoromethane sulfonic anhydride (20 mmol). The resulting mixture was stirred for 20 minutes keeping the temperature in the range of 18 to 22° C. until no starting material (I) was detected in the solution by TLC analysis. To the reaction mixture, 200 ml water (deionized) was added and the mixture was extracted 4 times with 100 ml ethyl acetate (in total 400 ml ethyl acetate). The combined organic extracts were dried over ca. 50 g sodium sulfate, filtered and evaporated to dryness to yield 15.1 g (86%) of Fipronil (II).

Reaction conditions (for example, the amount of EtOH used, reaction time, etc.), yield and purity can be optimized experimentally.

### Example 10

### Laboratory Scale Preparation of Compound of Formula II Using a Cyclohexylidenebishydroperoxyde System as Oxidizing Agent

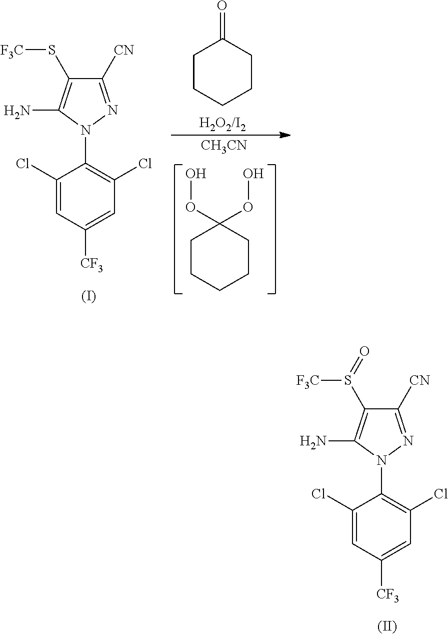

a) Preparation of Cyclohexylidenebishydroperoxyde

In a 0.5 liter 3-necked round bottom flask equipped with a dropping funnel, a reflux condenser, a mechanical stirrer, a thermometer and an inert gas supply, 1.02 g iodine (4 mmol) was dissolved under nitrogen in 200 ml acetonitrile and treated with 3.92 g cyclohexanone (40 mmol) and 18.1 ml 30% aqueous hydrogen peroxide (160 mmol). The resulting reaction mixture was stirred for 24 hours at room temperature. After completion of the reaction monitored by TLC, the solvent was removed under reduced pressure and 200 ml water (deionized) was added and the mixture was extracted 3 times with 200 ml dichloromethane (in total 600 ml dichloromethane). The combined organic layers were dried over 50 g sodium sulfate, filtered and evaporated to dryness to yield 5.50 g (93%) of reagent Cyclohexylidenebishydroperoxyde.

For reactions at larger scale, the safety aspects including the thermal stability of the reagent cyclohexylidenebishydroperoxyde should be thoroughly tested.

b) Oxidation Reaction of 2

In a 250 ml 3-necked round bottom flask equipped with a dropping funnel, a reflux condenser, a mechanical stirrer, a thermometer and an inert gas supply a solution of 8.42 g thioether (I) (20 mmol) in 150 ml dichloromethane was treated with 2.96 g cyclohexylidenebishydroperoxyde (20 mmol, as prepared under a). The reaction mixture was stirred for 60 minutes until all starting material (I) was reacted as evidenced by TLC analysis. After completion of the reaction, the reaction mixture was evaporated to dryness to yield 7.9 g (90%) Fipronil (II).

Reaction conditions (for example, reaction time, etc.), yield and purity can be optimized experimentally.

### Example 11

### Laboratory Scale Preparation of Compound of Formula II Using a can Catalyzed System as Oxidizing Agent

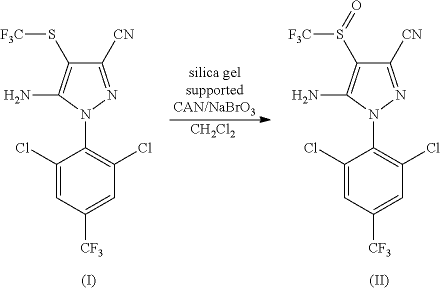

In a 0.5 liter 3-necked round bottom flask equipped with a dropping funnel, a reflux condenser, a mechanical stirrer, a thermometer and an inert gas supply, 50 g silica gel (dry) was treated dropwise in the course of 5 minutes with a solution of 1.10 g ceric ammonium nitrate (CAN, 2 mmol) and 3.32 g sodium bromate (NaBrO₃, 22 mmol) in 20 ml water (deionized) with vigorous stirring until a light yellow-orange colored, free flowing solid was obtained. After addition of 200 ml dichloromethane a solution of 8.42 g thioether (I) (20 mmol) in 50 ml dichloromethane was added dropwise over 10 minutes to the stirred heterogeneous mixture whereby the yellow-orange color disappeared instantaneously. The reaction mixture was stirred for 20 minutes until all starting material (I) was reacted as evidenced by TLC analysis. After completion of the reaction, the mixture was filtered and the filter cake was washed with 600 ml dichloromethane. The combined filtrates were evaporated to dryness to yield 7.9 g (90%) Fipronil (II).

Reaction conditions (for example, reaction time, etc.), yield and purity can be optimized experimentally.

### Example 12

### Laboratory Scale Preparation of Compound of Formula II Using a Gold(III) Catalyzed Oxidation

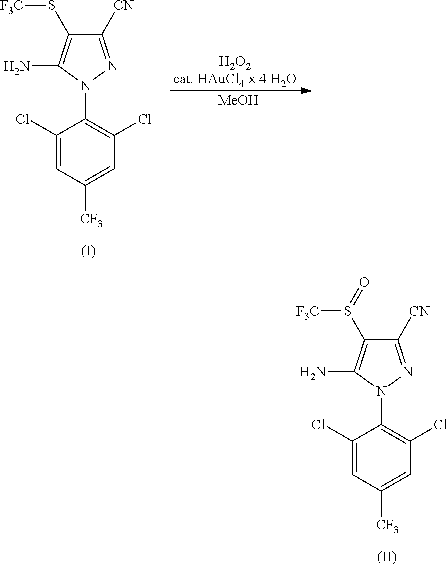

In a 200 ml 3-necked round bottom flask equipped with a dropping funnel, a reflux condenser, a mechanical stirrer, a thermometer and an inert gas supply, 8.42 g thioether (I) (20 mmol) in 10 ml methanol was treated under nitrogen with 82 mg hydrogen tetrachloroaurate(III) hydrate (HAuCl₄×4H₂O, 0.2 mmol) with stirring. To the reaction mixture, 4.08 ml 30% aqueous hydrogen peroxide (40 mmol) was added and the reaction mixture was stirred for 1 hour at room temperature until all starting material (I) disappeared as monitored by TLC. After completion of the reaction, the reaction mixture was extracted 3 times with 60 ml, in total with 180 ml ethyl acetate. The combined organic extracts were washed with 100 ml water (deionized), dried over ca. 50 g sodium sulfate, filtered and evaporated to dryness to yield 7.9 g (90%) Fipronil (II).

Reaction conditions (for example, reaction time, etc.), yield and purity can be optimized experimentally.

### Comparative Example 13

Direct sulfinylation of N-phenyl pyrazole starting material (III) according to known methods was tested. As such, sulfinylation was attempted using CF₃SO₂Na in the presence of a halogenating agent such as POCl₃, SOCl₂ or PBr₃.

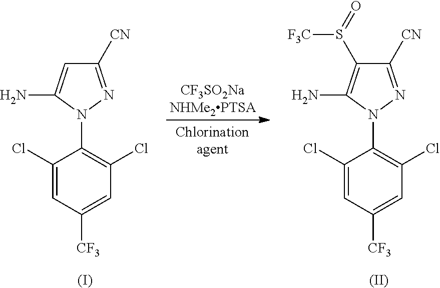

The reaction reagents and conditions tested are provided in Table I below.

TABLE I

| | | | | | | | | | | | | | | Reaction |
|---|---|---|---|---|---|---|---|---|---|---|---|---|---|---|
| | Compound (III) | | | CF₃SO₂Na | | | NHMe₂•PTSA | | | Reagent | | | Temp | Time |
| Batch No. | g | mmol | eq. | g | mmol | eq. | g | mmol | eq. | g | mmol | eq. | (° C.) | h |
| 1 | 16.30 | 50.76 | 1.00 | 15.85 | 101.56 | 2.00 | 16.50 | 75.94 | 1.50 | 10.00 g POCl₃ | 65.22 | 1.28 | 40 | 14 |
| 2 | 16.30 | 50.76 | 1.00 | 15.85 | 101.56 | 2.00 | 16.50 | 75.94 | 1.50 | 8.50 g SOCl₂ | 71.45 | 1.41 | 40 | 10 |
| 3 | 5.00 | 15.57 | 1.00 | 4.85 | 31.08 | 2.00 | 5.06 | 23.29 | 1.50 | 4.20 g PBr₃ | 15.52 | 1.00 | 57 ± 5 | 2 |
| 4 | 5.00 | 15.57 | 1.00 | 4.85 | 31.08 | 2.00 | 5.06 | 23.29 | 1.50 | 4.20 g PBr₃ | 15.52 | 1.00 | 0 | 6 |
| 5 | 5.50 | 17.13 | 1.00 | 5.35 | 34.28 | 2.00 | 5.60 | 25.77 | 1.50 | 4.65 g PBr₃ | 17.18 | 1.00 | −15 | 14 |

The results are provided in Table II below:

TABLE II

| Batch | Quantity | Yield | Purity, HPLC(%) |
|---|---|---|---|
| No. | g | % | (II) |
| 1 | 17.80 | 80.20 | 91.36 |
| 2 | 16.60 | 74.80 | 85.65 |

| | |
|---|---|
| 3 | Little product in the reaction mixture |
| 4 | Little product in the reaction mixture |
| 5 | Little product in the reaction mixture |

The reaction proceeded to the desired product, Fipronil, when SOCl₂ or POCl₃ were used as halogenating agents. However, PBr₃ did not yield the desired product, or at least not in acceptable yield (about 6%-8% (II) in the reaction mixture, according to HPLC).

### LIST OF REFERENCES

1. CN 1176078C
2. EP 0 668 269
3. EP 0374061
4. J-L. Clavel et al. in J. Chem. Soc. Perkin I, (1992), 3371-3375
5. Nicolaou, K. C.; Magolda, R. L.; Sipio, W. J.; Barnette, W. E.; Lysenko, Z.; Joullie, M. M., J. Am. Chem. Soc. 1980. 102, 3784
6. Khodaei et al., <<H₂O₂/Tf₂O System: An Efficient Oxidizing Reagent for Selective Oxidation of Sulfanes>>, Synthesis 2008 (11) 1682
7. Y. Venkateswarlu et al., <<A novel rapid sulfoxidation of sulfides with cyclohexylidenebishydroperoxide>> Tetrahedron Letters 2008 (49) 3463
8. Ali et al., <<Ceric Ammonium Nitrate Catalyzed Oxidation of Sulfides to Sulfoxides>>, Synthesis 2007 (22) 3507
9. Yu Yuan, Yubo Bian, <<Gold(III) catalyzed oxidation of sulfides to sulfoxides with hydrogen peroxide>> Tetrahedron Letters 2007 (48) 8518
10. S. B. Halligudi et al., <<One-step synthesis of SBA-15 containing tungsten oxide nanoclusters: a chemoselective catalyst for oxidation of sulfides to sulfoxides under ambient conditions>> Chem. Commun. 2007 4806
11. Remington's Pharmaceutical Sciences, Sixteenth Edition, E. W. Martin (Mack Publishing Co., Easton, Pa., 1980

1. A process for preparing fipronil comprising the steps of:
a) reacting CF₃S(═O)ONa with the compound of formula III 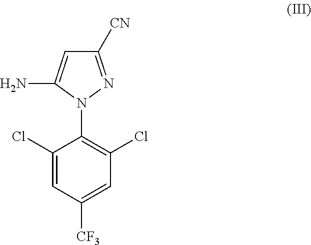
in the presence of a reducing/halogenating agent to produce the compound of formula I; and
b) oxidizing the compound of formula I obtained in step a) 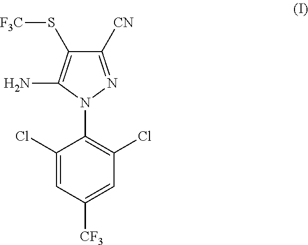
in the presence of a selective oxidizing agent, under suitable conditions, wherein the selective oxidizing agent selectively effects oxidation of the compound of formula I to fipronil.

2. The process of claim 1, wherein the selective oxidizing agent is H₂O₂/Tf₂O, cyclohexylidenebshydroperoxide, Ceric ammonium nitrate/sodium bromate, H₂O₂ in the presence of hydrogen tetrachloroaurate(III) hydrate, or MHSO₅ wherein M is an alkaline metal cation.

3. The process of claim 1, wherein the selective oxidizing agent is oxone (KHSO₅).

4. The process of claim 1, wherein the reducing/halogenating agent is PCl₃ or PBr₃.

5. The process of claim 1, wherein the reducing/halogenating agent is PCl₃.

6. The process of claim 1, wherein step a) of the process is carried out in the presence of a hydrochloride, methyl sulfonic acid (mesylate), benzene sulfonic acid or para-toluene sulfonic acid salt (tosylate) salt of a primary, secondary or tertiary amine.

7. The process of claim 6, wherein step a) of the process is carried out in the presence of dimethylamine tosylate salt.

8. The process of claim 1, wherein the selective oxidizing agent is KHSO₅ and, in step b), the compound of formula I and KHSO₅ are used in a molar ratio of compound or formula I to KHSO₅ ranging from 1.0 to 2.0.

9. The process of claim 1, wherein, in step b), oxone is added portionwise while maintaining the reaction temperature at about −10° C. in an organic acid as solvent.

10. The process of claim 1, wherein, in step b), the oxidation reaction is carried out at −15° C.±−3° C. in an organic acid as solvent.

11. The process of claim 10, wherein KHSO₅ is allowed to react with the compound of formula I for a time period ranging from 6 to 12 hours.

12. The process of claim 9, wherein the organic acid is trifluoroacetic acid.

13. The process of claim 1, wherein, in step b), the oxidation reaction is carried out at 25° C. to 30° C. in TFP as solvent.

14. The process of claim 13, wherein KHSO₅ is allowed to react with the compound of formula I for a time period ranging from 24 to 48 hours.

15. The process of claim 1, wherein step a) is carried out in the presence of a solvent selected from the group consisting of DMF, toluene, 2-methyl-tetrahydrofuran, and a mixture thereof.

16. Process for manufacturing an antiparasitic medicament comprising carrying out the process according to claim 1, and mixing the fipronil obtained by said process with a pharmaceutically acceptable carrier, adjuvant or vehicle.
